(19) United States
(12) Patent Application Publication
(10) Pub. No.: US 2015/0152114 A1
(43) Pub. Date: Jun. 4, 2015

(54) PROCESS FOR THE PREPARATION OF AN INTERMEDIATE FOR A TRIAZOLOPYRIMIDINE CARBONUCLEOSIDE

(71) Applicant: ENANTIA, S.L., Barcelona (ES)

(72) Inventors: Mireia Pastó Aguilá, Barcelona (ES); Alexander Christian Comely, Barcelona (ES)

(73) Assignee: ENANTIA, S.L., Barcelona (ES)

(21) Appl. No.: 14/419,865

(22) PCT Filed: Aug. 5, 2013
(86) PCT No.: PCT/EP2013/066356

§ 371 Date: Feb. 5, 2015

(30) Foreign Application Priority Data
Aug. 6, 2012 (ES) 201231273

(51) Int. Cl. C07D 487/04; C07D 239/52; C07D 239/42

(52) U.S. Cl. CPC C07D 487/04; C07D 239/42; C07D 239/52

(57) ABSTRACT

A process for the preparation of 4,6-dihalopyrimidin-5-amines of formula (II), or salts thereof, which comprises reacting 5-aminopyrimidin-4,6-diols of formula (III), or salts thereof, or a solvate either of the compound of formula (III) or of a salt thereof, with a halogenating agent, new intermediates useful in the preparation of the compound of formula (II) and processes for the preparation of these intermediates. The invention also refers to a process for the preparation of ticagrelor or a pharmaceutically acceptable salt thereof from 4,6-dihalo-2-(propylthio)pyrimidin-5-amine of formula (IIA).

The present invention refers to a process for the preparation of a compound of formula (II), to new intermediates useful for its preparation and to processes for the preparation of these intermediates. It also refers to the preparation of a compound of formula (I) and its pharmaceutically acceptable salts or its solvates or solvates of its salts by a process which comprises the preparation of a compound of formula (II).

### STATE OF THE ART

Ticagrelor is the name of the compound (1S,2S,3R,5S)-3-[7-[(1R,2S)-2-(3,4-difluorophenyl)cyclopropylamino]-5-(propylthio)-3H-(1,2,3)triazolo[4,5-d]pyrimidin-3-yl]-5-(2-hydroxyethoxy)cyclopentan-1,2-diol of formula (IA) whose chemical structure is the following.

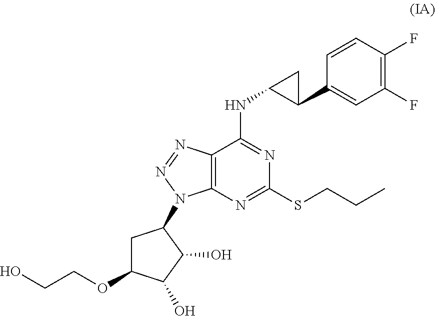 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor is a platelet aggregation inhibitor, in particular of the CYP3A4 receptor, and is indicated for the prevention of thrombotic events in patients suffering from acute coronary syndrome or myocardial infarction. Ticagrelor is absorbed rapidly after oral administration and is transformed into its principal metabolite by dehydroxyethylation at position 5 of the cyclopentane ring.

Patent application WO99/05143 discloses for the first time triazole[4,5-d]pyrimidine derivatives as P2T receptor antagonists, including among others ticagrelor, as well as a process for its preparation. This process is based on the incorporation of an amino group to the triazole[4,5-d]pyrimidine ring previously formed and subsequent transformation of the substituents of the lateral chains. The triazole[4,5-d]pyrimidine ring can be prepared by a diazotization reaction of a free amino group of a pyrimidine compound. The amino group is derived from a nitro group which is reduced using a metal catalyst.

Patent application WO2000/034283 also discloses triazole[4,5-d]pyrimidine derivatives including, among others, ticagrelor, where the configuration of all stereogenic centres is specified, as well as a process for its preparation. This process is the same as that described in the patent application WO99/05143.

Patent application WO2001/92263 discloses a preparation process for ticagrelor based on the preparation of the compound 4,6-dichloro-2-(propylthio)pyrimidin-5-amine of formula (IIA) by hydrogenolisis of a diazo compound followed by the subsequent preparation of the central ring of 1,2,3-triazol[4,5-d]pyrimidine according to the following synthetic scheme.

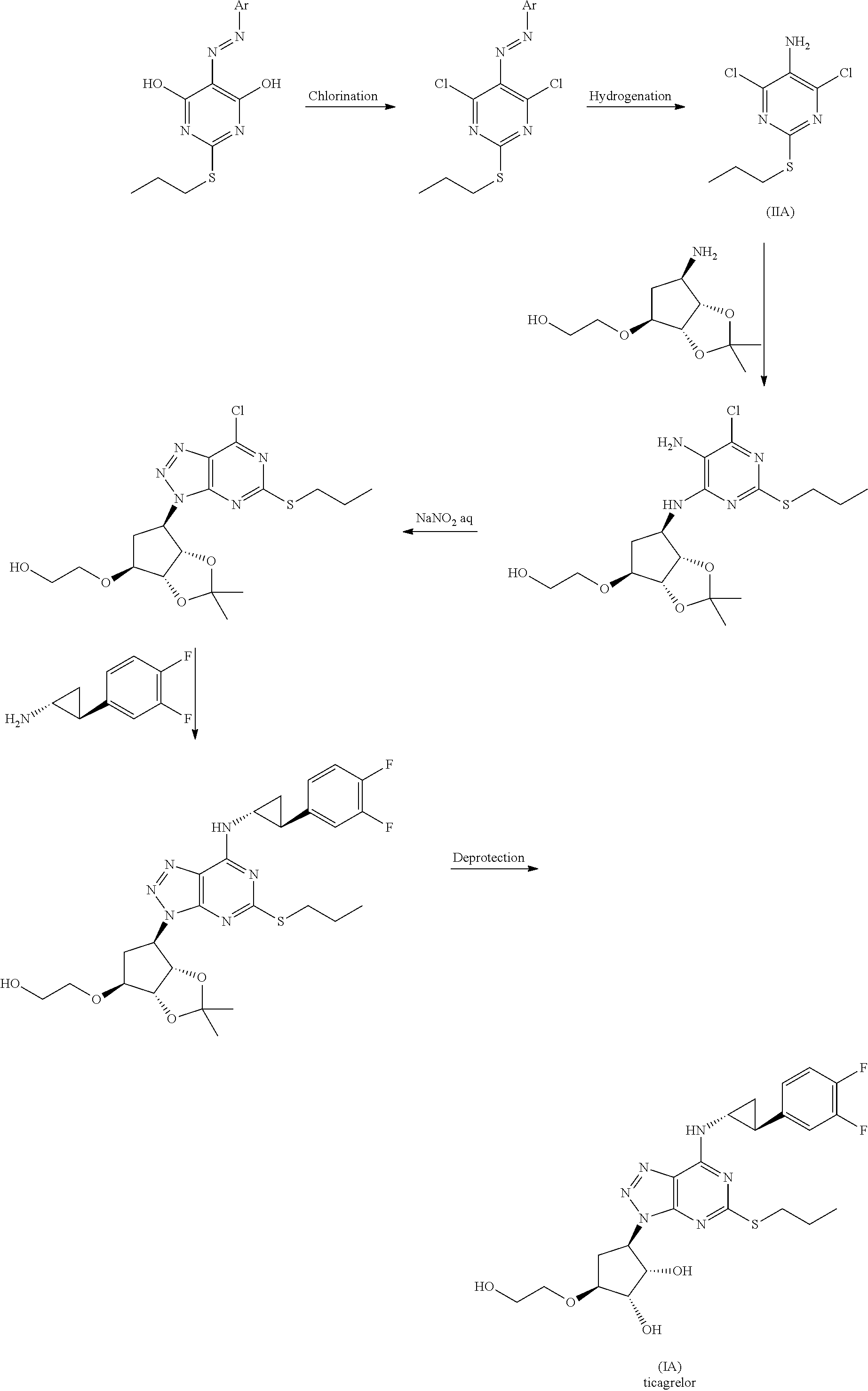

In certain steps of this process, in particular for the preparation of the compound 4,6-dichloro-2-(propylthio)pyrimidin-5-amine of formula (IIA), expensive starting materials and reaction conditions not convenient for use on an industrial scale are required.

Further processes for the preparation of the compound 4,6-dichloro-2-(propylthio)pyrimidin-5-amine of formula (IIA) in which the key step is a hydrogenation reaction have been disclosed. For example, in documents WO2005/095358 and WO2007/93369 a diazo group is hydrogenated to give the amino group of the compound of formula (IIA). In another process disclosed in WO2011/036479 a nitro group is hydrogenated to give the amino group of the compound of formula (IIA), according to the following scheme.

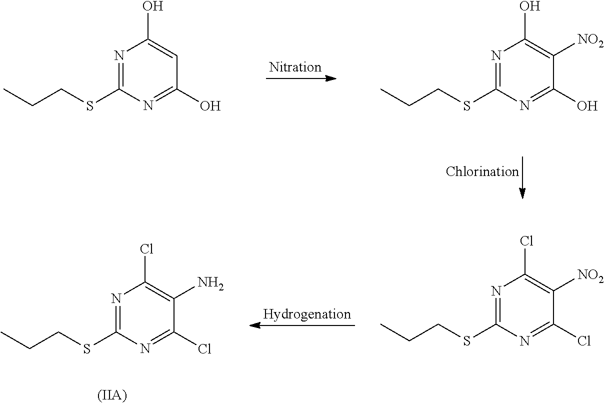

The industrial scale up of these processes causes considerable difficulties.

Another disclosed approach consists of the hydrogenolysis of the nitro group in a later step, after the reaction of the chloropyrimidine with the cyclopentylamine fragment, according to processes disclosed in patent applications WO9905153 and WO2011/017108.

Therefore, more economical and more easily industrializable processes would be of great interest for the preparation of the compound of formula (II) and, particularly, for the preparation of the compound of formula (IIA) which is useful for the preparation of ticagrelor.

### SUMMARY OF THE INVENTION

The inventors have found a new process for the preparation of the compound of formula (II) which comprises the use of low cost and commercially available starting materials and which presents several advantages, mainly in terms of yields, lower process costs, environmental impact, and at the same time allowing an easy industrialization.

These compounds are useful intermediates for the preparation of active pharmaceutical ingredients. In particular, the process of the present invention presents a different approach to the known processes for the preparation of 4,6-dichloro-2-(propylthio)pyrimidin-5-amine of formula (IIA) useful for the preparation of ticagrelor. It is based on the preparation of a pyrimidine ring which already incorporates a suitably protected amino group.

The remaining steps disclosed in the present invention also represent a significant improvement with respect to the procedures already disclosed. Moreover, when the different steps of the present invention are carried out together, the resulting process is a particularly efficient industrializable process.

In particular, when the compound of formula (II) is the compound of formula (IIA) where R is SCH₂CH₂CH₃, the process of the present invention comprises the formation of the ring of 4,6-dihydroxypyrimidine of formula (VA) by the condensation of low cost, commercially available starting materials; the alkylation of the thiol group to give the compound of formula (IVA); the deprotection of the amino group to give the compound of formula (IIIA), followed by the transformation of the two hydroxyl groups into the corresponding halogens to give the compound of formula (IIA). The process is carried out under mild, robust and selective conditions, and results in better yields than the processes disclosed in the state of the art.

Alternatively, when the compounds of formula (II) are those where R is selected from the group consisting of H, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl, the process of the invention comprises the formation of the ring of 4,6-dihydroxypyrimidine of formula (V) by the condensation of the corresponding starting materials which are low cost and commercially available compounds; the deprotection of the amino group to give the compound of formula (III), followed by the transformation of the two hydroxyl groups into the corresponding halogens to give the compound of formula (II).

Furthermore, the process of the present invention proceeds through new intermediates also forming part of the invention.

Therefore, an aspect of the present invention is to provide a process for the preparation of a compound of formula (II) or a salt thereof,

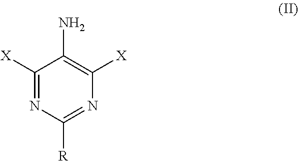

wherein X is a halogen selected from the group consisting of chlorine, bromine and iodine, R is selected from the group consisting of H, SR′, NHR′, N(R)₂, CH₃, CH₂R, CH(R′)₂, and C(R′)₃; and each R′ is independently selected from the group consisting of (C₁-C₅)alkyl, aryl, and (C₁-C₅)alkylaryl; which comprises reacting a compound of formula (III) or a salt thereof, or a solvate either of the compound of formula (III) or of a salt thereof,

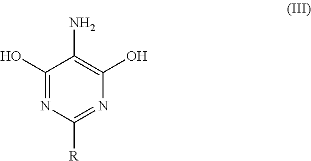

with a halogenating agent at a temperature comprised from 70 to 140° C.

In a particular embodiment of the process of the invention is prepared a compound of formula (IIA) or a salt thereof,

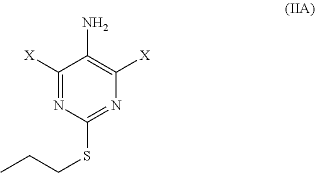

wherein X is a halogen selected from the group consisting of chlorine, bromine and iodine, which comprises reacting the compound of formula (IIIA) or a salt thereof,

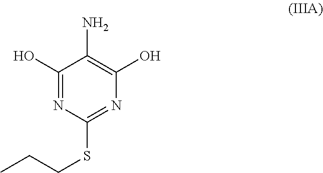

with a halogenating agent at a temperature comprised from 70 to 140° C.

In another particular embodiment of the process of the invention is prepared a compound of formula (IIA)

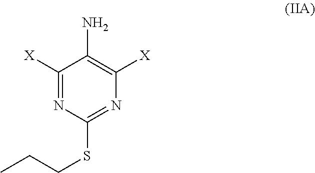

wherein X is a halogen selected from the group consisting of chlorine, bromine and iodine, which comprises reacting the solvate of the compound of formula (IIIA) or the solvate of the salt thereof,

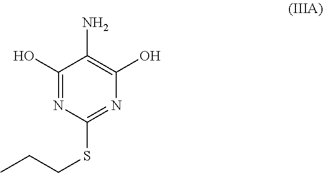

with a halogenating agent at a temperature comprised from 70 to 140° C.

A halogenating agent is understood to be any compound or element which contains at least one active halogen which can activate an organic compound and transfer at least one halide.

In a preferred embodiment of the process of the invention, in the compound of formula (IIA), X is chlorine and the halogenating agent is a chlorinating agent; preferably, the chlorinating agent is selected from the group consisting of POCl₃, PCl₃, PCl₅, SOCl₂ and SO₂Cl₂or mixtures thereof; more preferably, POCl₃.

In another preferred embodiment, in the compound of formula (IIA), X is bromine and the halogenating agent is a brominating agent; preferably, the brominating agent is selected from the group consisting of POBr₃ and PBr₃.

In another preferred embodiment, the reaction is carried out at a temperature comprised from 90 to 110° C.; preferably, at 100° C.

In another preferred embodiment, the reaction is carried out at a pressure comprised from atmospheric pressure to 10 Bar; preferably, comprised from 1 to 6 Bar. The term “atmospheric pressure” refers to the force per unit area exerted on a surface by the weight of air above that surface at a given location. For the purpose of this description, atmospheric pressure is comprised from 0.7 and 2.0 Bar.

Optionally, the reaction can be carried out in an appropriate solvent. Appropriate solvents for the preparation of a compound of formula (IIA) from a compound of formula (IIIA) can be (C₃-C₆)ethers such as methyl tert-butyl ether, 2-methyltetrahydrofuran or tetrahydrofuran; halogenated (C₁-C₆)alkyl solvents such as dichloromethane; aromatic (C₆-C₉)alkyl solvents such as toluene or xylene; (C₅-C₁₂)alkanes such as cyclohexane or heptane; or mixture thereof.

The process for the preparation of a compound of formula (IIA) from a compound of formula (IIIA) can also be carried out in the presence of a small amounts of a solvent selected from (C₁-C₆)alcohol such as methanol, ethanol, or iso-propanol; (C₃-C₉)ketones such as acetone, methyl ethyl ketone or methyl isobutyl ketone; water; and mixtures thereof. Generally, the amount of the above-mentioned solvents with respect to the total mixture is equal to or less than 5% by weight.

Optionally, the reaction can be carried out in the presence of a catalyst, wherein this catalyst is selected from the group consisting of an amide or a quaternary ammonium halide salt. Examples of appropriate amides as catalysts in the present invention can be, among others, dimethylformamide. Examples of appropriate quaternary ammonium halide salts as catalysts in the present invention can be, among others, tetrabutylammonium chloride.

Optionally, the reaction can be carried out in the presence of a base; preferably, in the presence of an organic base such as N,N-diethylaniline.

Analogously, a compound of formula (II) wherein R is selected from the group consisting of H, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl can be prepared from a compound of formula (III) according to the process defined above for the compound of formula (IIIA) using the same type of solvents and reaction conditions.

The compounds of formula (II) and (III) are basic, so that salts thereof can be prepared by reaction with non-toxic acids, including inorganic and organic acids. Appropriate acids for the formation of salts of compound (III) are, among others, benzenesulfonic acid, benzoic acid, fumaric acid, hydrobromic acid, hydrochloric acid, maleic acid, methanesulfonic acid, phosphoric acid, succinic acid, sulfuric acid and p-toluenesulfonic acid.

A solvate of a compound of formula (III) or a salt thereof can be prepared by dispersing the compound of formula (III) or a salt thereof or a solvate either of the compounds of formula (III) or of a salt thereof, in an appropriate solvent. Appropriate solvents for the preparation of a solvate of the compound of formula (III) or a salt thereof include, among others, water, (C₁-C₆)alcohol, (C₃-C₉)ketone and (C₄-C₁₀)ethers and their mixtures with water. Examples of suitable (C₃-C₉)ketones can be acetone, methyl ethyl ketone, methyl isobutyl ketone, cyclohexanone and cyclopentanone. Examples of suitable alcohols can be methanol, ethanol, isopropanol and isobutanol. Examples of suitable ethers can be tetrahydrofuran, tert-butylmethylether and dioxane.

In a preferred embodiment, the solvate of the compound of formula (IIIA) or a salt thereof is prepared by dispersing the compound (IIIA) or a salt thereof or a solvate either of the compound of formula (IIIA) or of a salt thereof, in an appropriate solvent as defined above for the compound of formula (III) or a salt thereof or a solvate either of the compound of formula (III) or of a salt thereof.

Alternatively, a solvate of a compound of formula (IIIA) or of a salt thereof can be directly obtained by the reaction of the compound of formula (IVA) with an acid or a base as defined below. The appropriate solvents used in the preparation of the compound of formula (IIIA) from the compound of formula (IVA) are defined below. Analogously, a solvate of a compound of formula (III) wherein R is selected from the group consisting of R is selected from the group consisting of H, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl can be directly obtainable by the reaction of the compound of formula (V) with an acid or a base as defined below.

In a preferred embodiment, the compound of formula (III) is the compound of formula (IIIA). In another preferred embodiment, the compound of formula (III) is that where R is selected from CH₃ and H.

In an embodiment, the process of the invention further comprises an additional step of desolvating the compound of formula (III). Desolvating techniques are widely known in the state of the art. A suitable desolvating step for the present invention comprises a drying step; preferably the desolvating step further comprises the following steps: suspension in a solvent which does not form a solvate, a distillation, and filtration step. The desolvating step can be carried out at a temperature comprised from 40 to 100° C., and under a pressure comprised form atmospheric pressure to vacuum.

The term “solvate” refers to a molecular complex comprising the compound of formula (III) or a salt thereof, and a stoichiometric or non-stoichiometric amount of one or more solvent molecules bound by non-covalent intermolecular forces. When the one or more solvent molecules forming part of the molecular complex is water, the solvate is a hydrate. Preferably, the molar ratio between moles of the solvate molecules and moles of the compound of formula (III) is comprised from 0.2:1 to 1:5; more preferably comprised from 0.5:1 to 1:2.

In a preferred embodiment, the preparation process of a compound of formula (IIA) comprises reacting a salt of the compound of formula (IIIA) with a halogenating agent as described previously; preferably, the salt of the compound of formula (IIIA) is 5-amino-2-(propylthio)pyrimidin-4,6-diol hydrochloride (IIIA.HCl). In an embodiment of the invention, the salt of the compound of formula (IIIA.HCl) is a solvate.

The compound of formula (IIIA) or a salt thereof, or a solvate either of the compound of formula (IIIA) or of a salt thereof, are key process intermediates. These compounds are new and also form part of the invention. In a preferred embodiment, the salt of the compound of formula (IIIA) is 5-amino-2-(propylthio)pyrimidin-4,6-diol hydrochloride (IIIA.HCl).

The compound of formula (III) or a salt thereof, or a solvate either of the compound of formula (III) or of its salts, can be obtained in high yield and chemical purity from a compound of formula (IV).

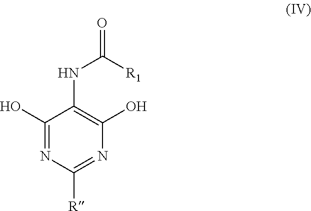

In an embodiment of the invention, when in the compound of formula (III) R is SR′; and R′ is selected from the group consisting of (C₁-C₅)alkyl, aryl, and (C₁-C₅)alkylaryl, the process further comprises a previous step wherein a compound of formula (IV),

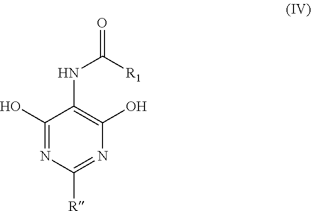

wherein R″ is SR′; R′ is selected from the group consisting of (C₁-C₅)alkyl, aryl, and (C₁-C₅)alkylaryl; and R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl, is reacted with an acid or a base at a temperature comprised from 40 to 150° C.

In a particular embodiment, the compound of formula (IIIA) or a salt thereof, or a solvate either of the compound of formula (IIIA) or of its salts, can be obtained in high yield and chemical purity by a process which comprises reacting a compound of formula (IVA)

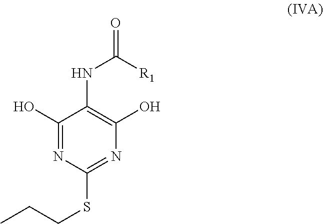

wherein R₁ is a radical selected from the group consisting of (C₁-C₅)alkyl, phenyl, (C₁-C₅)alkylphenyl, H, O(C₁-C₅)alkyl, O(C₁-C₅)alkylphenyl and O-phenyl, with an acid or a base in an appropriate solvent at a temperature comprised from 40 to 150° C.; preferably, at a temperature comprised from 40 to 100° C.

The term alkyl refers to a linear or branched hydrocarbon chain which contains the number of carbon atoms specified in the description or the claims. Preferably, the alkyl group is selected from methyl, isopropyl and tert-butyl.

The term alkylaryl refers to a group resulting from the replacement of a hydrogen atom of an alkyl group, defined previously, with an aryl group.

The term alkylphenyl refers to a group resulting from the replacement of a hydrogen atom of an alkyl group, defined previously, with a phenyl group. Preferably, the alkylphenyl group is benzyl.

The term O-alkyl refers to a linear or branched hydrocarbon chain which contains the number of carbon atoms specified in the description or the claims bound to the carbonyl group through an oxygen atom. Preferably, the O-alkyl group is selected from methoxy, ethoxy, isopropoxy, and tert-butoxy.

The term O-alkylphenyl refers to a group resulting from the replacement of a hydrogen atom of an O-alkyl group, defined previously, with a phenyl group. Preferably, the O-alkylphenyl group is benzyloxy.

In a preferred embodiment, the previous procedure comprises reacting the compound of formula (IVA) wherein R₁ is a radical selected from the group consisting of methyl and O-tert-butyl with an acid at a temperature comprised from 40 to 100° C.; preferably, R₁ is methyl and the reaction is carried out at a temperature comprised from 50 to 70° C.; preferably, at 50° C.

The reaction is carried out in the presence of an acid, wherein the acid can be an inorganic or an organic acid. Appropriate acids for the preparation of the compound of formula (IIIA) from a compound of formula (IVA) are, among others, hydrochloric acid, hydrobromic acid, nitric acid, perchloric acid, sulfuric acid, trifluoroacetic acid and formic acid; preferably, the acid is hydrochloric acid.

In another preferred embodiment, the previous procedure comprises reacting the compound of formula (IVA) wherein R₁ is selected from the group consisting of (C₁-C₅)alkyl, phenyl, (C₁-C₅)alkylphenyl, H, O(C₁-C₅)alkyl, O(C₁-C₅)alkylphenyl and O-phenyl with a base at a temperature comprised from 40 to 100° C. in an appropriate solvent.

The reaction is carried out in the presence of a base. Appropriate bases for the preparation of the compound of formula (IIIA) from the compound of formula (IVA) are, among others, sodium hydroxide, potassium hydroxide and barium hydroxide.

Alternatively, the previous procedure comprises submitting the compound of formula (IVA) wherein R₁ is benzyloxy to hydrogenolysis in an appropriate solvent.

The preparation of the compound of formula (IIIA) from the compound of formula (IVA) is carried out in an appropriate solvent. Appropriate solvents for the preparation of the compound of formula (ÎIIA) from the compound of formula (IVA) are those solvents which are water miscible such as water, (C₁-C₆)alcohol, (C₃-C₉)ketone, and tetrahydrofuran; preferably, the solvent is a (C₁-C₆)alcohol selected from the group consisting of methanol, ethanol and isopropanol.

Analogously, the process for preparing the compound of formula (III) comprises reacting the compound of formula (IV) wherein R″ is SR′ with an acid or a base; according to the process defined above for the compound of formula (IVA) using the same reaction conditions. Appropriate acids, bases, and solvents for the preparation of the compound of formula (III) from a compound of formula (IV) wherein R″ is SR′ are the same as those mentioned above for the compound of formula (IVA).

The compounds of formula (IVA) can be prepared by a process which comprises reacting a compound of formula (VA),

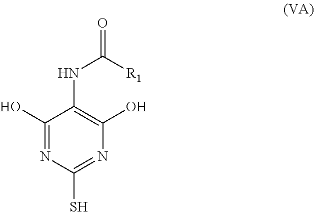

with a compound of formula CH₃CH₂CH₂Y (VIA), wherein Y is a leaving group selected from the group consisting of chloride, bromide, iodide and —SO₂R₃, wherein R₃ is selected from a group consisting of (C₁-C₅)alkyl and (C₅-C₁₈)aryl, in the presence of a base in an appropriate solvent.

Analogously, the compounds of formula (IV) wherein R″ is SR′ can be prepared by a process which comprises reacting a compound of formula (V),

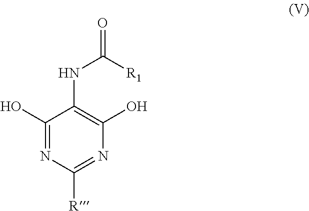

wherein R′″ is SH with a compound of formula R′Y (VI), wherein R′ is selected from the group consisting of (C₁-C₅)alkyl, aryl, and (C₁-C₅)alkylaryl; and Y is a leaving group selected from the group consisting of chloride, bromide, iodide and —OSO₂R₃, wherein R₃ is selected from a group consisting of (C₁-C₅)alkyl and (C₅-C₁₈)aryl, in the presence of a base in an appropriate solvent.

The term aryl refers to a radical of a ring system with 1, 2 or 3 rings, the rings being aromatic and being isolated or totally or partially fused having 5 or 6 ring members, being each of the members independently selected from C, CH, N, NH, O or S, the rings being chemically possible. The ring system is optionally substituted by one or more radicals independently selected from the group consisting of (C₁-C₆)alkyl, O(C₁-C₆)alkyl, nitro, cyano and halogen.

In a preferred embodiment, the process for the preparation of a compound of formula (IVA) is carried out when, in the compound of formula (VIA), Y is bromine.

The transformation of a compound of formula (VA) into a compound of formula (IVA) by reaction with a compound of formula (VIA) is carried out in the presence of a base. Appropriate bases are, among others, metal hydroxides such as sodium hydroxide, potassium hydroxide or calcium hydroxide; alkaline and alkaline earth metal carbonates such as sodium carbonate and potassium carbonate; organic tertiary amines such as triethylamine; metal hydrides such as sodium hydride, potassium hydride or calcium hydride; and (C₁-C₄)alkoxides of alkaline or alkaline earth metals such as sodium methoxide, sodium ethoxide or potassium tert-butoxide. In a preferred embodiment, the base is a metal hydroxide selected from the group consisting of sodium hydroxide, potassium hydroxide, or calcium hydroxide; preferably, the base is sodium hydroxide.

The transformation of a compound of formula (VA) into a compound of formula (IVA) is carried out in the presence of an appropriate solvent. Appropriate solvents for the present invention are those which are water miscible, such as (C₁-C₆)alcohol and tetrahydrofuran; preferably, the solvent is a (C₁-C₆)alcohol selected from the group consisting of methanol, ethanol and isopropanol.

Analogously, the transformation of a compound of formula (V) wherein R′″ is SH into a compound of formula (IV) wherein R″ is SR′ is carried out by reaction with the compound of formula (VI) according to the process defined above for the compound of formula (VA) using the same reaction conditions. Appropriate bases and solvents are the same mentioned above for the compound of formula (VA).

The compounds of formula (IVA) are key process intermediates obtained in high yield and high chemical purity. The compounds of formula (IVA) wherein R₁ is a radical selected from the group consisting of (C₁-C₅)alkyl, phenyl, (C₁-C₅)alkylphenyl, H, O(C₁-C₅)alkyl, O(C₁-C₅)alkylphenyl and O-phenyl are new and also form part of the invention. In a preferred embodiment, the compound of formula (IVA) is that wherein R₁ is a radical selected from the group consisting of —H, methyl, —O-tert-butyl and —O-benzyl; more preferably, the compound of formula (IVA) is that wherein R₁ is methyl.

Analogously, the compounds of formula (IV) wherein R″ is SR′, and R′ is selected from the group consisting of (C₁-C₅)alkyl, aryl, and (C₁-C₅)alkylaryl are key intermediates obtained in high yield and high chemical purity. The compounds of formula (IV) wherein R″ is SR′, R′ is selected from the group consisting of (C₁-C₅)alkyl, aryl, and (C₁-C₅)alkylaryl, and R₁ is a radical selected from the group consisting of (C₁-C₅)alkyl, phenyl, (C₁-C₅)alkylphenyl, H, O(C₁-C₅)alkyl, O(C₁-C₅)alkylphenyl and O-phenyl are new and also form part of the invention.

Analogously, the compounds of formula (V) are key intermediates obtained in high yield and high chemical purity. The compound of formula (V) wherein R′″ is H; and R₁ is CH₃ is new and also forms part of the invention.

Alternatively, the compounds of formula (III) wherein R is selected from the group consisting of H, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl can be directly prepared from the compound of formula (V) where R′″ is selected from the group consisting of H, SR′, NHR′, N(R′)₂, CH₃, CH₂R′, CH(R′)₂, and C(R′)₃. Thus, in an embodiment of the process of the invention, a compound of formula (V) wherein R′″ is selected from the group defined above; and wherein R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl, is reacted with an acid or a base in an appropriate solvent at a temperature comprised from 40 to 100° C. to give the compound of formula (III).

The reaction can be carried out in the presence of an acid, wherein the acid can be an inorganic or an organic acid. Appropriate acids for the preparation of the compound of formula (III) from a compound of formula (V) can be, among others, hydrochloric acid, hydrobromic acid, nitric acid, perchloric acid, sulfuric acid, trifluoroacetic acid and formic acid; preferably, the acid is hydrochloric acid.

Alternatively, the reaction can be carried out in the presence of a base. Appropriate bases for the preparation of the compound of formula (III) from the compound of formula (V) can be, among others, sodium hydroxide, potassium hydroxide or barium hydroxide.

The transformation of a compound of formula (V) into a compound of formula (III) is carried out in the presence of an appropriate solvent. Appropriate solvents for the present invention are those solvents which are water miscible such as water, (C₁-C₆)alcohol, (C₃-C₉)ketone, and tetrahydrofuran; preferably, the solvent is a (C₁-C₆)alcohol selected from the group consisting of methanol, ethanol and isopropanol.

In a preferred embodiment, the transformation of a compound of formula (V) into a compound of formula (III) is carried out by reacting with an acid or a base at a temperature comprised from 40 to 100° C.; preferably, R₁ is methyl and the reaction is carried out at a temperature comprised from 50 to 80° C.; preferably, at 60° C.

The compounds of formula (V) can be prepared by a process which comprises reacting a compound of formula (VII),

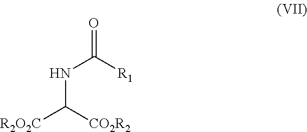

wherein each R₂ is independently a (C₁-C₅)alkyl radical, with a compound of formula (VIII)

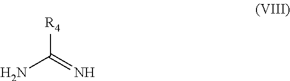

wherein R₄ is selected from the group consisting of H, SH, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl); in the presence of a base in an appropriate solvent at a temperature comprised from 60° C. to reflux temperature of the solvent and, subsequently, the compound obtained is treated with an acid to give the compound of formula (V).

In a preferred embodiment, the compounds of formula (V) can be prepared by a process which comprises reacting a compound of formula (VII) with a compound of formula (VIII) at a temperature comprised from 50° C. to reflux temperature of the solvent.

In a preferred embodiment, the compound of formula (V) is a compound of formula (VA). The compounds of formula (VA) can be prepared by a process which comprises reacting a compound of formula (VII),

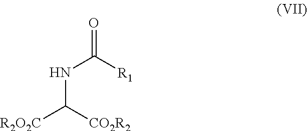

wherein each of R₂ is independently (C₁-C₅)alkyl, with the compound of formula (VIIIA),

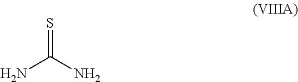

in the presence of a base in an appropriate solvent at a temperature comprised from 60° C. to reflux temperature of the solvent, and subsequent treatment of the compound obtained with an acid.

The compound of formula (VIII) wherein R₄ is SH, and the compound of formula (VIIIA) are tautomers of thiourea. The term “tautomer” or “tautomeric forms” refers to constitutional isomers of the same organic compound wherein a hydrogen atom or proton migrates accompanied by a switch of a single bond and adjacent double bond.

The term “reflux temperature” refers to the temperature at which the mixture boils under conditions in which the solvent vapor returns to the liquid mixture after condensation.

In a preferred embodiment, the process for the preparation of a compound of formula (V) is carried out when, in the compound of formula (VII) each R₂ is ethyl; preferably, in the compound of formula (VII) R₁ is H, methyl, O-tert-butyl, O-benzyl and each R₂ is ethyl; more preferably, in the compound of formula (VII) R₁ is methyl and each R₂ is ethyl.

In a preferred embodiment, the process for the preparation of a compound of formula (VA) is carried out when, in the compound of formula (VII) each R₂ is ethyl; preferably, in the compound of formula (VII) R₁ is H, methyl, O-tert-butyl, O-benzyl and each R₂ is ethyl; more preferably, in the compound of formula (VII) R₁ is methyl and each R₂ is ethyl.

The coupling reaction between a compound of formula (VII) and the compound of formula (VIIIA) is known in the state of the art (cf. Harnden, et al. “The chemistry of Pyridinethiols. III* The synthesis of some substituted pyridinethiols and some thiazolo[5,4-d]pyrimidines”, Aust. J. Chem. 1990, vol. 43, pp. 55-62). This reaction, for example, is carried out in an appropriate solvent such as water miscible solvents such as a (C₁-C₆)alcohol such as methanol and ethanol and tetrahydrofuran, at an appropriate temperature, preferably at reflux temperature of the solvent. The reaction is carried out in the presence of a base. Appropriate bases for the preparation of the compound of formula (V) can be, among others, (C₁-C₄)alkoxides of alkaline or alkaline earth metals such as sodium methoxide, sodium ethoxide or potassium tert-butoxide; preferably, sodium methoxide and sodium ethoxide. Subsequently, the obtained compound is treated with an acid such as hydrochloric acid.

Individual steps or a combination of steps for the preparation of a compound of formula (II) from a compound of formula (V) are considered part of the invention. A process which comprises the preparation of a compound of formula (V) as described previously, and one or more steps of the process for the preparation of a compound of formula (II), are also part of the invention. Particularly, individual steps or a combination of steps for the preparation of a compound of formula (IIA) from a compound of formula (VA) are considered part of the invention. A process which comprises the preparation of a compound of formula (VA) as described previously, and one or more steps of the process for the preparation of a compound of formula (IIA), are also part of the invention.

Another aspect of the invention relates to a process for the preparation of a compound of formula (II) defined previously, further comprising transforming the compound of formula (II) into a pharmaceutically active ingredient of general formula (I) and optionally transforming the compound of formula (I) into a pharmaceutically acceptable salt thereof

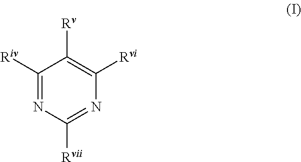

with the proviso that:
(a) R^{iv} and R^{v} form, together with the C atoms to which they are bound, the 5 membered heterocycle of formula
   R^{vi} is

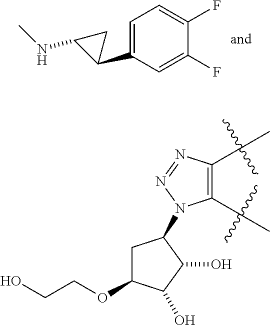 [SMILES: CN[C@@H]1C[C@H]1c1ccc(F)c(F)c1 ; *c1nnn([C@@H]2C[C@H](OCCO)[C@@H](O)[C@H]2O)c1*]

R^{vii} is —SCH₂CH₂CH₃ ; or alternatively
(b) R^{iv} and R^{v} form, together with the C atoms to which they are bound, the 5 membered heterocycle of formula

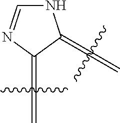 [SMILES: *=c1nc[nH]c1=*]

R^{vi} is SH; and R^{vii} is H; or alternatively,
(c) R^{iv} and R^{v} form, together with the C atoms to which they are bound, the 5 membered heterocycle of formula

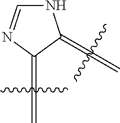 [SMILES: *=c1nc[nH]c1=*]

R^{vi} is

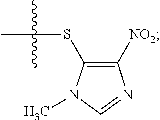 [SMILES: *Sc1c([N+](=O)[O-])ncn1C]

and
   R^{vii} is H; or alternatively,
(d) R^{iv}—OCH₃; R^{v} is

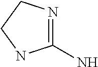

R^{vi} is Cl; and R^{vii} is CH₃.

In an embodiment, the compound of formula (I) is mercaptopurine of formula (IB). Mercaptopurine is the name of the compound 3,7-dihydropurine-6-thione wherein R^{iv} and R^{v} form, together with the C atoms to which they are bound, the 5 membered heterocycle of formula

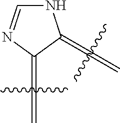 [SMILES: *=c1nc[nH]c1=*]

R^{vi} is SH; and R^{vii} is H of formula (IB)

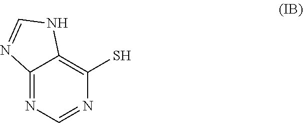

In an embodiment, the compound of formula (I) is azathioprine of formula (IC). Azathioprine is the name of the compound 6-[(1-methyl-4-nitro-1H-imidazol-5-yl)sulfanyl]-7H-purine wherein R″ and R″ form, together with the C atoms to which they are bound, the 5 membered heterocycle of formula

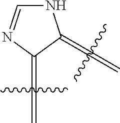 [SMILES: *=c1nc[nH]c1=*]

R^{vi} is

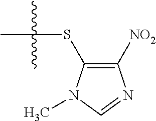 [SMILES: *Sc1c([N+](=O)[O-])ncn1C]

and R^{vi} is H of formula (IC)

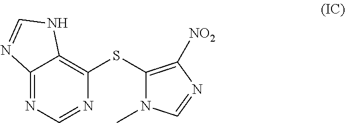 [SMILES: Cn1cnc([N+](=O)[O-])c1Sc1ncnc2nc[nH]c12]

In an embodiment, the compound of formula (I) is moxonidine of formula (ID). Moxonidine is the name of the compound 4-chloro-N-(4,5-dihydro-1 H-imidazol-2-yl)-6-methoxy-2-methylpyrimidin-5-amine wherein R^{iv}—OCH₃; R^{v} is

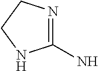

R^{vi} is Cl; and R^{vii} is CH₃ of formula (ID)

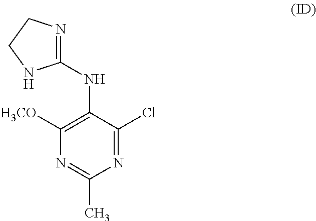 [SMILES: COc1nc(C)nc(Cl)c1NC1=NCCN1]

Particularly, the invention relates to a process for the preparation of a compound of formula (IIA) defined previously; further comprising transforming the compound of formula (IIA) into a compound of formula (IA) and optionally transforming the compound of formula (IA) into a pharmaceutically acceptable salt thereof. The compound of formula (IA) is a compound of formula (I) wherein R^{iv} and R^{v} form, together with the C atoms to which they are bound, the 5 membered heterocycle of formula

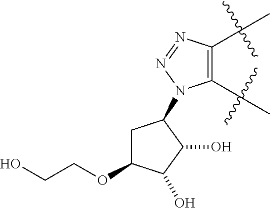 [SMILES: *c1nnn([C@@H]2C[C@H](OCCO)[C@@H](O)[C@H]2O)c1*]

R^{vi} is

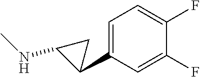

and

R^{vii} is —SCH₂CH₂CH₃.

The term “pharmaceutically active ingredient” or “active drug compound” used in the present invention refers to a compound with proved pharmaceutical activity demonstrated in clinical trials and approved as a drug by any Medicine Agency such as the European Medicines Agency (EMEA) or the US Food and Drug Administration (FDA).

The term “pharmaceutically acceptable salt” used in the present invention refers to any salt formed from pharmaceutically non-toxic acids, including organic and inorganic acids. There is no limitation with respect to these salts, except that, if used for therapeutic purposes, they must be pharmaceutically acceptable.

Since the compound of formula (I), particularly the compound of formula (IA), is a basic compound, its salts can be prepared from pharmaceutically non-toxic acceptable acids, including organic and inorganic acids. Such acids include benzenesulfonic acid, benzoic acid, ethanesulfonic acid, fumaric acid, hydrobromic acid, hydrochloric acid, maleic acid, methanesulfonic acid, phosphoric acid, succinic acid, sulfuric acid, and p-toluenesulfonic acid.

Generally, such salts are prepared reacting those compounds with a stoichiometric amount of the appropriate acid in water, or an organic solvent, or a mixture thereof.

The compound of formula (I), particularly the compound of formula (IA) can be in crystalline form, either non-solvated or as a solvate (for example, hydrates) and it is intended that both forms are within the scope of the present invention. Solvation methods are generally known in the state of the art. The preparation of pharmaceutically acceptable salts of the compound of formula (I), particularly the compound of formula (IA), can be carried out by methods known in the state of the art.

In a preferred embodiment, the transformation of a compound of formula (IIA) into the compound of formula (IA) comprises the following steps:

(a) reacting a compound of formula (IIA)

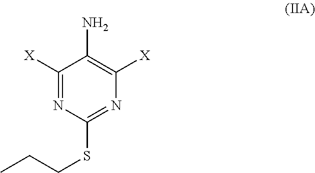

wherein X is a halogen selected from the group consisting of chlorine, bromine and iodine with a compound of formula (IX) or a salt thereof;

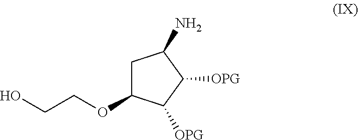

wherein PG is an alcohol protecting group, to give the compound of formula (XA)

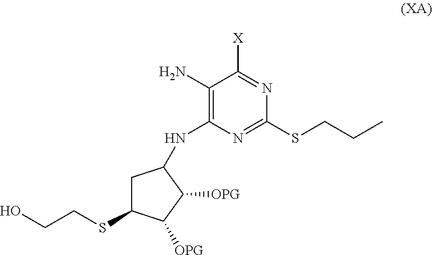

(b) reacting the compound of formula (XA) obtained in step (a) with an alkaline metal nitrite in the presence of an acid to give the compound of formula (XIA)

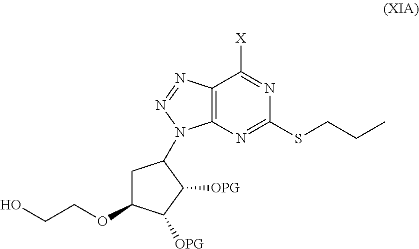

(c) reacting the compound of formula (XIA) obtained in step (b) with a compound of formula (XII)

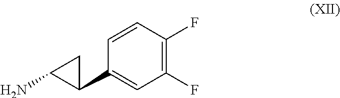 [SMILES: N[C@@H]1C[C@H]1c1ccc(F)c(F)c1]

to give the compound of formula (XIIIA)

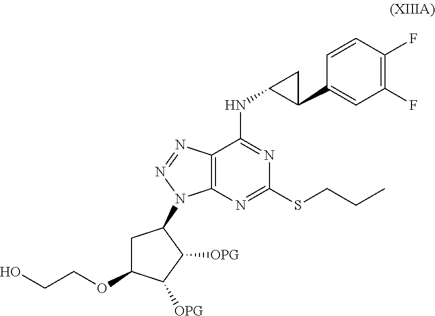

and (d) deprotecting the compound of formula (XIIIA) obtained in step (c) to give the compound of formula (IA).

In a preferred embodiment, in the transformation of a compound of formula (IIA) into the compound of formula (IA) as defined previously, X is chlorine.

Appropriate alcohol protecting groups (PG) for the present invention can be ester forming protecting groups such as acetyl or tert-butylcarbonyl; ether forming protecting groups, such as methoxymethyl, methoxyethoxymethyl, tetrahydropyranyl, benzyl, p-methoxybenzyl or triphenylmethyl; silyl ether forming protecting groups such as trimethylsilyl, triisopropylsilyl, tert-butyldimethylsilyl, [2-(trimethylsilyl)ethoxy]methyl; and ketals formed from reagents such as acetone, 2,2-dimethoxypropane, 2-methoxy-1-propene and cyclohexanone. In a particular embodiment, the compound (IX) is the compound of formula (IX).

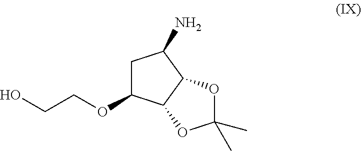

The compound of formula (XIA) can be prepared by reaction of a compound of formula (XA) with a nitrite. Appropriate nitrites for the present invention can be, among others, alkaline and alkaline earth metal nitrites such as sodium nitrite or potassium nitrite; or organic nitrites such as isoamyl nitrite. This reaction is carried out in the presence of an acid such as acetic acid and in the presence of an appropriate solvent such as water or a (C₁-C₅)alcohol.

The compounds of formula (II) can be used in the preparation of other compounds with pharmacological activity. U.S. Pat. No. 4,323,570 discloses a compound of formula (II) where R is CH₃ useful for the preparation of antihypertensives and in the treatment of glaucoma.

The compounds of formula (IIA) can be used in the preparation of other compounds with pharmacological activity. U.S. Pat. No. 5,654,285 discloses a compound of formula (IIA) useful for the preparation of platelet aggregation inhibitors.

Throughout the description and claims, the word “comprising” and its variations are not intended to exclude other technical characteristics, additives, components or steps. For a skilled person in the art, other objects, advantages and features of the invention will be evident partially from the description and partially from the practice of the invention. The following examples and drawings are provided by way of illustration and are not intended to be limiting of the present invention. The numerical signs relating to the drawings and placed in brackets in a claim are only intended to improve the understanding of the claim and are not to be interpreted as limiting to the scope of protection of the claim. Furthermore, the present invention covers all possible combinations of particular and preferred embodiments described herein.

### EXAMPLES

The following abbreviations have been used in the examples.

Ac: Acetyl

AcOEt: Ethyl acetate

Ar: Argon

c.: Concentrated

CBz: Benzyloxycarbonyl

DMF: Dimethylformamide

Et₃N: Triethylamine

EtOH: Ethanol

IPA: Isopropanol

RT: Room temperature

THF: Tetrahydrofuran

TLC: Thin Layer Chromatography

General Considerations

The proton nuclear magnetic resonance spectra were recorded on a Varian Mercury 400 spectrometer in DMSO-d6. HPLC/MS were recorded on an Agilent 6100 Single Quadrupole LC/MS System using an Xbridge C18 XP 30×4.6 mm, 2.5 μm column.

### Example 1

### Preparation of N-(4,6-dihydroxy-2-mercaptopyrimidin-5-yl)acetamide (Compound of Formula VA wherein R₁ is methyl)

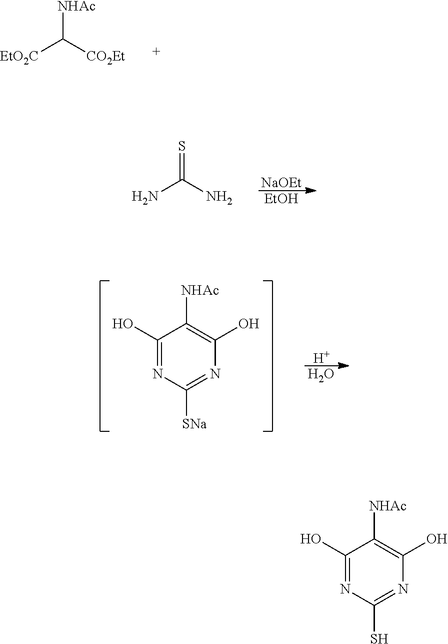

Procedure with Isolation of the Sodium Salt.

Diethylacetamidomalonate (5 g, 23.01 mmol) and thiourea (2.45 g, 32.2 mmol, 1.4 eq) were added to a solution of sodium ethoxide (21% w/w, 20 ml, 52.9 mmol, 2.3 eq) in EtOH (65 mL). The reaction mixture was heated to reflux temperature for 4 h. The resulting suspension was cooled to RT, then to 0° C., was filtered and the sodium solid salt was washed with EtOH (10 mL).

The sodium salt was dissolved in a minimum amount of water (25 mL) and the solution was acidified to pH 1 with concentrated HCl (4 mL). The resulting precipitate was filtered and was washed with cold EtOH (5 mL) and cold Et₂O (5 mL). The solid was dried under vacuum providing N-(4,6-dihydroxy-2-mercaptopyrimidin-5-yl)acetamide (3.24 g, 70% yield, 100% HPLC-MS) as a yellowish solid.

¹H-NMR (DMSO-d6, 400 MHz, ppm): 12.35 (s, 2H, OH), 9.17 (s, 1H, NH), 2.00 (s, 3H, CH₃).

Procedure Without Isolation of the Sodium Salt

Diethylacetamidomalonate (5 g, 23.01 mmol) and thiourea (2.45 g, 32.2 mmol, 1.4 eq) were added to a solution of sodium ethoxide (21% w/w, 20 ml, 52.9 mmol, 2.3 eq) in EtOH (30 mL). The reaction mixture was heated to reflux temperature for 3 h. The resulting suspension was cooled to RT and then to 0° C. Water (25 mL) was added followed by concentrated. HCl (4 ml) to reach pH 1. The resulting precipitate was filtered and was washed with cold EtOH (5 mL) and cold Et₂O (5 mL) providing N-(4,6-dihydroxy-2-mercaptopyrimidin-5-yl)acetamide (3.2 g, 68% yield) as a yellowish solid.

### Example 2

### Preparation of N-(4,6-dihydroxy-2-(propylthio)pyrimidin-5-yl)acetamide (Compound of Formula IVA Wherein R₁ is Methyl)

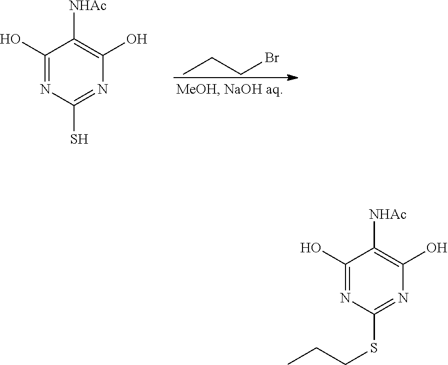

Aqueous NaOH (50% w/w, 2.6 mL, 50 mmol, 5 eq) was slowly added at 0° C. to a suspension of N-(4,6-dihydroxy-2-mercaptopyrimidin-5-yl)acetamide of example 1 (2 g, 9.95 mmol) in MeOH (10 mL). The reaction mixture was stirred at RT for 30 min and 1-bromopropane (2.6 mL, 30 m mol, 3 eq) was added dropwise, the resulting solution was stirred at RT overnight and a solid precipitated. The solvent was evaporated and water (7 mL) was added to obtain a clear solution. Concentrated HCl (2.4 mL) was added and the resulting solid was filtered, was washed with cold water (2 mL) and was dried by vacuum providing N-(4,6-dihydroxy-2-(propylthio)pyrimidin-5-yl)acetamide (1.96 g, 82% yield, 97.8% HPLC-MS) as an off-white solid.

¹H-NMR (DMSO-d6, 400 MHz, ppm): 12.54 (s, 1H, OH), 11.44 (s, 1H, OH), 8.87 (s, 1 H, NH), 3.06 (t, J=6.8 Hz, 3H, CH₂), 1.94 (s, 3H, CH₃), 1.64 (sex, J=6.8 Hz, J=7.6 Hz, 2H, CH₂), 1.02 (t, J=7.6 Hz, 3H, CH₃).

### Example 3

### Preparation of 5-amino-2-(propylthio)pyrimidine-4,6-diol Hydrochloride (Compound IIIA-HCl)

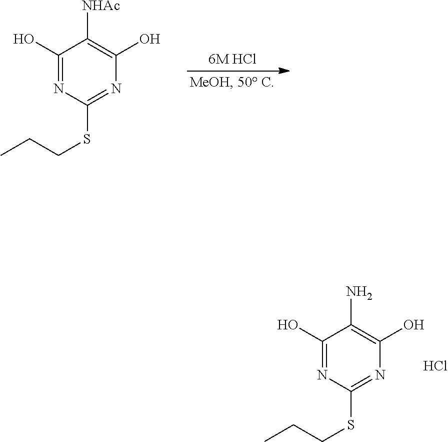 [SMILES: CCCSc1nc(O)c(NC(C)=O)c(O)n1 ; CCCSc1nc(O)c(N)c(O)n1 ; Cl]

6 M HCl (5 mL) was added to a suspension of N-(4,6-dihydroxy-2-(propylthio)pyrimidin-5-yl)acetamide of example 2 (5 g, 20.55 mmol) in MeOH (20 mL). The resulting suspension was stirred at 50° C. for 18 h and was evaporated to dryness. Toluene was added to the residue and the mixture was concentrated to dryness (3×20 mL) providing 5-amino-2-(propylthio)pyrimidine-4,6-diol (III-HCl) (5 g, quantitative yield, 93.5% HPLC-MS) as a yellowish solid

¹H-NMR (DMSO-d6, 400 MHz, ppm): 3.10 (t, J=6.8 Hz, 3H, CH₂), 1.65 (sex, J=6.8, 7.6 Hz, 2H, CH₂), 0.95 (t, J=7.6 Hz, 3H, CH₃).

### Example 4

### Preparation of 4,6-dichloro-2-(propylthio)pyrimidine-5-amine (Compound of Formula IIA Wherein X is a Chlorine)

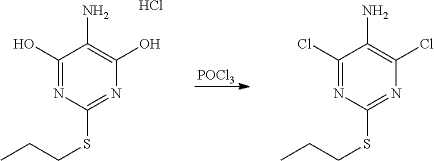

Procedure A:

In a sealed tube, 5-amino-2-(propylthio)pyrimidine-4,6-diol (IIIA-HCl) of Example 3 (200 mg, 0.84 mmol) and POCl₃ (2.5 mL) were mixed and the mixture was heated at reflux temperature for 22.5 h. Excess of POCl₃ was evaporated at reduced pressure. Water (5 mL) and EtOAc (5 mL) were added, and the layers were separated. The aqueous layer was extracted with EtOAc (5 mL), and the combined organic layers were dried over MgSO₄, were filtered and were evaporated providing 4,6-dichloro-2-(propylthio)pyrimidine-5-amine (192 mg, 96% yield, 100% HPLC-MS) as a brown oil.

Procedure B:

In a sealed tube, 5-amino-2-(propylthio)pyrimidine-4,6-diol (IIIA-HCl) of Example 3 (1 g, 4.2 mmol) and POCl₃ (5 mL) were mixed and the mixture was heated at reflux temperature for 6.5 h. Excess of POCl₃ was evaporated under reduced pressure. Water (10 mL) and EtOAc (10 mL) were added, and the layers were separated. The organic layer was washed with water (10 mL) and the aqueous layer was extracted with EtOAc (10 mL), the combined organic layers were washed with saturated aqueous NaHCO₃ (10 mL), were dried over MgSO₄, were filtered and were evaporated providing 4,6-dichloro-2-(propylthio)pyrimidine-5-amine (541 mg, 54% yield, 100% HPLC-MS) as a dark oil.

¹H-NMR (DMSO-d6, 400 MHz, ppm): 5.85 (s, 2H, NH₂), 2.97 (t, J=6.8 Hz, 3H, CH₂), 1.63 (sex, J=6.8, 7.6 Hz, 2H, CH₂), 0.94 (t, J=7.6 Hz, 3H, CH₃).

### Example 5

### Preparation of 5-amino-2-(propylthio)pyrimidine-4,6-diol Hydrochloride Isopropanol Solvate (Compound IIIA-HCl-IPA)

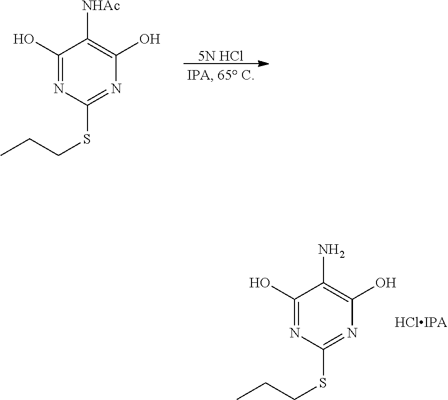

In a 250 mL flask equipped with magnetic stirring was prepared a suspension of N-(4,6-dihydroxy-2-(propylthio)pyrimidin-5-yl)acetamide of example 2 (10.0 g, 41.1 mmol) in IPA (60 mL). 5 N HCl in IPA (20 mL) was added dropwise over about 10 min and the reaction mixture was stirred at 65° C. for 19 h. The resulting suspension was cooled to 20° C. and was stirred at this temperature for 2 h. After that time, the suspension was filtered and the solid was washed with IPA (20 mL) providing 5-amino-2-(propylthio)pyrimidine-4,6-diol hydrochloride isopropanol solvate (IIIA-HCl-IPA) (11.0 g, 85%, 24 wt % IPA) as a white solid.

¹H-NMR (DMSO-d6, 400 MHz, ppm): 3.75 (sept, 0.86H, J=6.0 Hz), 3.12 (t, 2H, J=7.2 Hz), 1.66 (sext., 2H, J=7.2 Hz), 1.01 (d, 5.16H, J=6.0 hz), 0.96 (t, 3H, J=7.2 Hz).

### Example 6

### Preparation of 4,6-dichloro-2-(propylthio)pyrimidine-5-amine (Compound of Formula IIA Where X is a Chlorine)

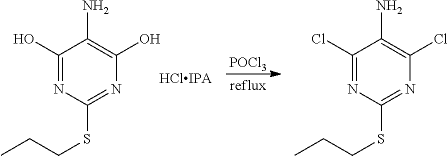

In a sealed tube, a solution of 5-amino-2-(propylthio)pyrimidine-4,6-diol isopropanol solvate (IIIA-HCl-IPA) of Example 5 (500 mg, 0.84 mmol, 20 wt % IPA) and POCl₃ (5 mL) were heated at reflux temperature for 15 h. Excess POCl₃ was removed on a rotary evaporator and water (10 mL) and toluene (5 mL) were added. The layers were separated and the aqueous layer was extracted with toluene (2.5 mL). The combined organic layers were washed with saturated NaHCO₃ (5 mL), H₂O (2.5 mL), dried over MgSO₄, and filtered. The mixture thus obtained was evaporated under reduced pressure providing 4,6-dichloro-2-(propylthio)pyrimidine-5-amine (319 mg, 79% yield) as a dark red oil.

### Example 7

### Preparation of N-(4,6-dihydroxypyrimidin-5-yl)acetamide (Compound of Formula V Where R is H; and R₁ is COCH₃)

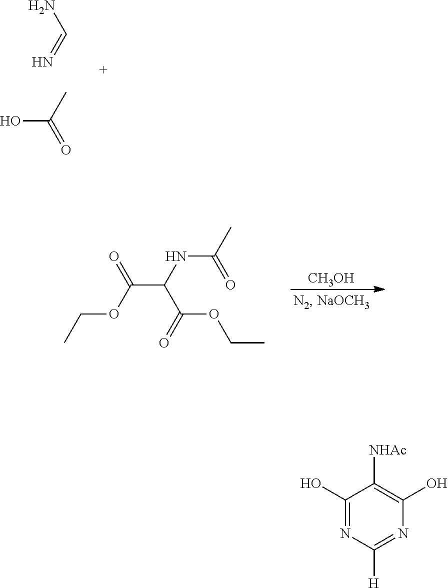

Formamidine acetate (19.11 g, 184 mmol) and anhydrous MeOH (200 mL) were combined under N₂. The mixture was cooled to 0° C. and a solution of NaOMe (25 wt % in MeOH, 168 mL, 734 mmol,) was added over 15 min and was stirred for 15 min. Diethyl 2-acetamidomalonate (40 g, 184 mmol) was added and the reaction mixture was stirred at reflux temperature for 2 h. The black suspension was cooled to 0° C., was stirred for 15 min, was filtered and the solid was washed with cold MeOH (40 mL) providing N-(4,6-dihydroxypyrimidin-5-yl)acetamide (47.9 g) as a grey solid.

¹H-NMR (D₂O, 400 MHz, ppm): 7.79 (s, 1H), 2.13 (s, 3H).

### Example 8

### Preparation of 5-aminopyrimidine-4,6-diol (Compound of Formula III Where R is H; and R₁ is H)

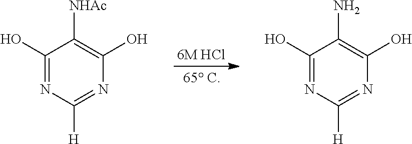 [SMILES: CC(=O)Nc1c(O)ncnc1O ; Nc1c(O)ncnc1O]

To N-(4,6-dihydroxypyrimidin-5-yl)acetamide (40 g) of Example 7 in MeOH (200 mL) was added 6 M HCl (160 mL) and the reaction mixture was stirred at 65° C. for 15 h. The resulting suspension was cooled to 20° C. and was stirred at this temperature for 30 min. The suspension was filtered and the solid was washed with MeOH (40 mL) providing 5-aminopyrimidine-4,6-diol (33.5 g) as a white solid. The mother liquors were cooled to 0° C. and the resulting suspension was filtered and the solid was washed with MeOH (5 mL) providing 5-aminopyrimidine-4,6-diol (3.5 g) as a beige solid.

¹H-NMR (D₂O, 400 MHz, ppm): 8.77 (s, 1 H).

### Example 9

### Preparation of 4,6-dichloropyrimidin-5-amine (Compound of Formula II Where R is H; X is Chlorine; and R₁ is H)

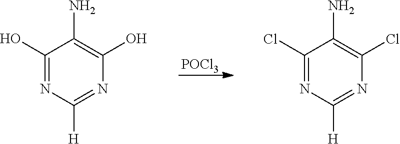

In a sealed tube was introduced POCl₃ (4 mL) and 5-aminopyrimidine-4,6-diol (0.4 g) of Example 8, the system was purged with argon and was stirred at 110° C. for 48 h. The mixture was cooled to RT and excess POCl₃ was removed by rotary evaporation. The residue was mixed with CH₂Cl₂ (2 mL) and H₂O (1 mL) was added at 0° C. Saturated aqueous K₂CO₃ was added to pH 7/8 and the resulting suspension was filtered. The liquid layers were separated and organic layer was dried over Na₂SO₄ and concentrated to provide 4,6-dichloropyrimidin-5-amine (44 mg).

¹H-NMR (CDCl₃, 400 MHz, ppm): 8.21 (s, 1H).

### Example 10

### Preparation of N-(4,6-dihydroxy-2-methylpyrimidin-5-yl)acetamide (Compound of Formula V Where R is CH₃; and R₁ is COCH₃)

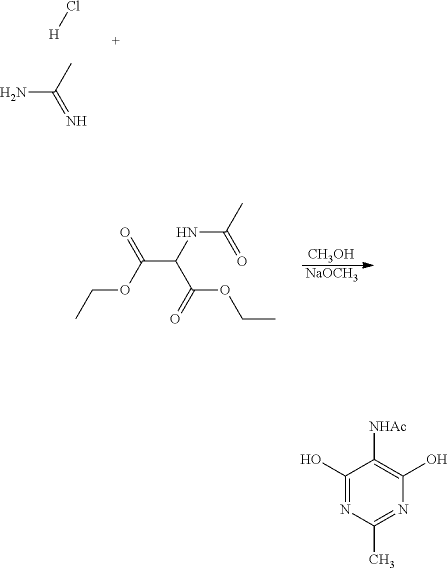

To diethyl 2-acetamidomalonate (2.00 g, 9.21 mmol) and acetamidine hydrochloride (1.22 g, 12.89 mmol) cooled to 0° C. was added a solution of NaOMe (25 wt % in MeOH, 6.95 mL, 30.4 mmol). The reaction mixture was stirred at reflux temperature for 4 h. Further MeOH (25 mL) was added to obtain a stirrable mixture and the reaction was stirred at reflux temperature for 12 h. The mixture was cooled to RT, the resulting suspension was filtered and the solid was washed with cold MeOH (2×6 mL) and dried providing N-(4,6-dihydroxy-2-methylpyrimidin-5-yl)acetamide (1.95 g) as a yellow solid.

¹H-NMR (D₂O, 400 MHz, ppm): 2.25 (s, 2H), 2.13 (s, 3H).

### Example 11

### Preparation of 5-amino-2-methylpyrimidine-4,6-diol (Compound of Formula III Where R is CH₃; and R₁ is H)

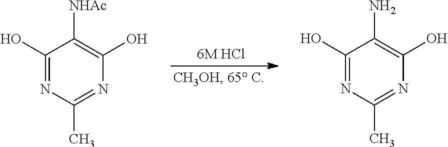

To N-(4,6-dihydroxy-2-methylpyrimidin-5-yl)acetamide (1.8 g) of Example 10 in MeOH (9 mL) was added 6 M HCl (7.2 mL) and the reaction mixture was stirred at 50° C. for 15 h under argon. The resulting suspension was cooled to 20° C. and was stirred at this temperature for 30 min. The suspension was filtered and the solid was washed with cold MeOH (2×2 mL) providing 5-amino-2-methylpyrimidine-4,6-diol (1.21 g) as an off-white solid.

¹H-NMR (DMSO-d6, 400 MHz, ppm): 2.38 (s, 3H).

### Example 12

### Preparation of 4,6-dichloro-2-methylpyrimidin-5-amine (Compound of Formula II Where R is H; X is Chlorine; and R₁ is H)

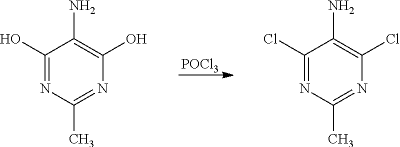

In a sealed tube was introduced 5-amino-2-methylpyrimidine-4,6-diol (500 mg) of Example 11 and POCl₃ (2.5 mL) and the system was purged with argon and was stirred at 110° C. for 20 h. The mixture was cooled to RT and was poured onto ice (5 mL). CH₂Cl₂ (5 mL) was added and the pH was adjusted to 8 with 8 M NaOH. The layers were separated and the aqueous layer was extracted with CH₂Cl₂ (1.5 mL) The combined organic layers were washed with H₂O (2×2.5 mL) and were evaporated under reduced pressure providing 4,6-dichloro-2-methylpyrimidin-5-amine (129 mg, 26% yield) as a brown solid.

¹H-NMR (DMSO-d6, 400 MHz, ppm): 6.00-5.62 (bs, 2H), 2.40 (s, 3H).

### REFERENCES CITED

WO99/05143

WO2000/034283

WO2001/92263

WO2011/036479

WO2007/93368

WO2005/095358

Harnden, et al. “The chemistry of Pyridinethiols. III* The synthesis of some substituted pyridinethiols and some thiazolo[5,4-d]pyrimidines”. Aust. J. Chem. 1990, vol. 43, pp. 55-62.

WO2011/017108

U.S. Pat. No. 5,654,285

U.S. Pat. No. 4,323,570

1. A process for the preparation of a compound of formula (II) or a salt thereof, 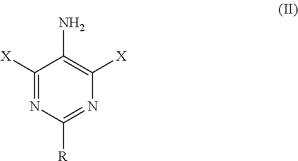
wherein X is a halogen selected from the group consisting of chlorine, bromine and iodine; R is selected from the group consisting of H, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl; which comprises reacting a compound of formula (III) or a salt thereof, or a solvate either of the compound of formula (III) or of a salt thereof, 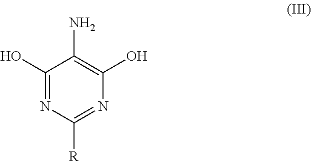
with a halogenating agent at a temperature comprised from 70 to 140° C.

2. The process according to claim 1 wherein, when in the compound of formula (III) R is SR′; and R′ is selected from the group consisting of (C₁-C₅)alkyl, aryl, and (C₁-C₅)alkylaryl, the process further comprises a previous step wherein a compound of formula (IV), 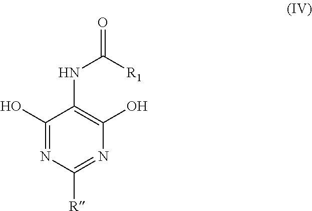
wherein R″ is SR′; R′ is selected from the group consisting of (C₁-C₅)alkyl, aryl, and (C₁-C₅)alkylaryl; and R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl, is reacted with an acid or a base at a temperature comprised from 40 to 150° C. to give the compound of formula (III) wherein R is SR′.

3. The process according to claim 2, further comprising a previous step wherein a compound of formula (V) 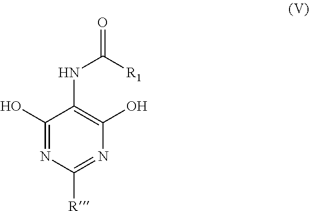
wherein R′″ is SH and R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl, is reacted with a compound of formula R′Y (VI) wherein R′ is selected from the group consisting of (C₁-C₅)alkyl, aryl, and (C₁-C₅)alkylaryl; and Y is a leaving group selected from the group consisting of chlorine, bromine, iodine and —OSO₂R₃, wherein R₃ is a radical selected from the group consisting of (C₁-C₅)alkyl and (C₅-C₁₈)aryl, in the presence of a base in an appropriate solvent to give the compound of formula (IV) wherein R″ is SR′.

4. The process according to claim 1, wherein the compound of formula (II) is the compound of formula (IIA) and the compound of formula (III) is the compound of formula (IIIA) or a salt thereof;

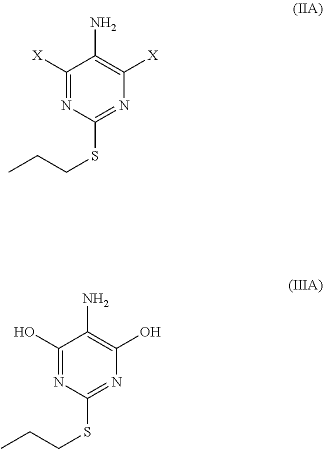

5. The process according to claim 4, wherein one or both:
in the compound of formula (IIA) X is chlorine and the halogenating agent is a chlorinating agent; or
the salt of the compound of formula (IIIA) is 5-amino-2-(propylthio)pyrimidin-4,6-diol hydrochloride (IIIA-HCl).

6. (canceled)

7. The process according to claim 4, further comprising a previous step wherein a compound of formula (IVA), 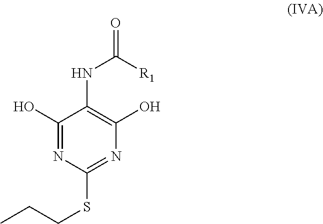
wherein R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl, is reacted with an acid or a base in an appropriate solvent at a temperature comprised from 40 to 150° C. to give the compound of formula (IIIA).

8. The process according to claim 7, wherein R₁ is a radical selected from the group consisting of methyl and —O-tert-butyl and the reaction is carried out by reacting a compound of formula (IVA) with an acid at a temperature comprised from 40 to 100° C.

9. The process according to claim 7, wherein the acid is hydrochloric acid.

10. The process according to claim 7, further comprising a previous step wherein a compound of formula (VA) 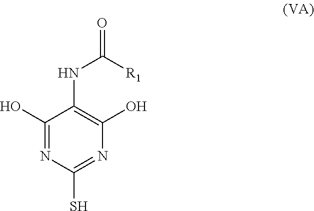
wherein R is SH; and R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl, is reacted with a compound of formula CH₃CH₂CH₂Y (VIA) wherein Y is a leaving group selected from the group consisting of chlorine, bromine, iodine and —OSO₂R₃, wherein R₃ is a radical selected from the group consisting of (C₁-C₅)alkyl and (C₅-C₁₈)aryl, in the presence of a base in an appropriate solvent to give the compound of formula (IVA).

11. The process according to claim 10, wherein one or both:
Y is bromine or,
the base is NaOH.

12. (canceled)

13. The process according to claim 1, further comprising a previous step wherein a compound of formula (V), 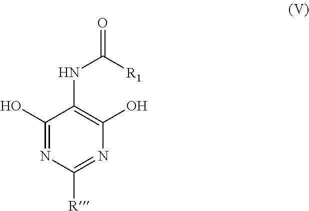
wherein R′″ is selected from the group consisting of H, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl); and R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl, is reacted with an acid or a base in an appropriate solvent at a temperature comprised from 40 to 150° C. to give the compound of formula (III).

14. The process according to claim 10, further comprising a previous step wherein a compound of formula (VII), 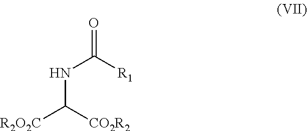
wherein each R₂ is independently a (C₁-C₅)alkyl radical, is reacted with a compound of formula (VIII) 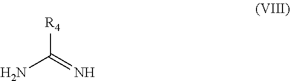
wherein R₄ is selected from the group consisting of H, SH, SR′, NHR′, N(R′)₂, CH₃, CH₂R′, CH(R′)₂, and C(R′)₃; and each R′ is independently selected from the group consisting of (C₁-C₅)alkyl, aryl, and (C₁-C₅)alkylaryl); in the presence of a base in an appropriate solvent at a temperature comprised from 60° C. to reflux temperature of the solvent and, subsequently, the compound obtained is treated with an acid to give the compound of formula (V).

15. The process according to claim 14, wherein the compound of formula (V) is the compound of formula (VA), and the compound of formula (VIII) is the compound of formula (VIIIA) 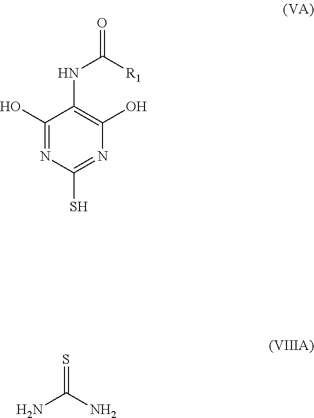
wherein in the compound of formula (VA) R is SH and R₁ is a radical selected from the group consisting of —(C₁-C₅)alkyl, -phenyl, —(C₁-C₅)alkylphenyl, —H, —O(C₁-C₅)alkyl, —O(C₁-C₅)alkylphenyl and O-phenyl; and in the compound of formula (VIIIA) R₄ is S.

16. The process according to claim 15, wherein one or both:
in the compound of formula (VII) each R₂ is ethyl or,
in the compound of formula (VII) R₁ is methyl.

17. (canceled)

18. The process according to claim 1, further comprising one or both:
transforming a compound of formula (II) into a pharmaceutically active ingredient of formula (I) and,
transforming the compound of formula (I) into a pharmaceutically acceptable salt thereof; 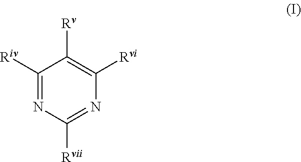
with the proviso that:
(a) R^{iv} and R^{v} form, together with the C atoms to which they are bound, the 5 membered heterocycle of formula 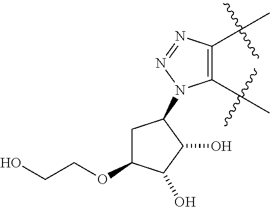 [SMILES: *c1nnn([C@@H]2C[C@H](OCCO)[C@@H](O)[C@H]2O)c1*]
R^{vi} is 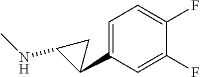
and
R^{vii} is —SCH₂CH₂CH₃ ; or alternatively
(b) R^{iv} and R^{v} form, together with the C atoms to which they are bound, the 5 membered heterocycle of formula 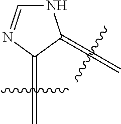 [SMILES: *=c1nc[nH]c1=*]
R^{vi} is SH; and R^{vii} is H; or alternatively,
(c) R^{iv} and R^{v} form, together with the C atoms to which they are bound, the 5 membered heterocycle of formula 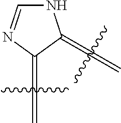 [SMILES: *=c1nc[nH]c1=*]
R^{vi} is 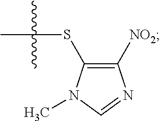 [SMILES: *Sc1c([N+](=O)[O-])ncn1C]
and
R^{vii} is H; or alternatively,
(d) R^{iv}—OCH₃; R^{v} is 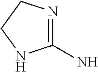
R^{vi} is Cl; and R^{vii} is CH₃.

19. The process according to claim 18, wherein the compound of formula (I) is the compound of formula (IA) 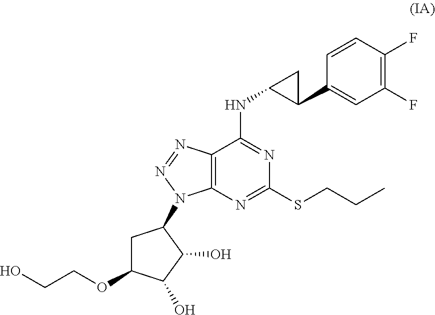
wherein R^{iv} and R^{v} form, together with the C atoms to which they are bound, the 5 membered heterocycle of formula
R^{vi} is 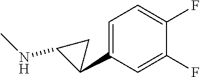
and 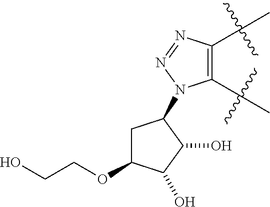 [SMILES: *c1nnn([C@@H]2C[C@H](OCCO)[C@@H](O)[C@H]2O)c1*]
R^{vii} is —SCH₂CH₂CH₃.

20. A compound of formula (IIIA) or a salt thereof, or a solvate either of the compound of formula (IIIA) or of a salt thereof as defined in claim 4.

21. The compound according to claim 20, which is a solvate of 5-amino-2-(propylthio)pyrimidin-4,6-diol hydrochloride or alternatively; which is 5-amino-2-(propylthio)pyrimidin-4,6-diol hydrochloride (IIIA-HCl).

22. (canceled)

23. A compound of formula (IVA) as defined in claim 7, wherein R₁ is a radical selected from the group consisting of (C₁-C₅)alkyl, phenyl, (C₁-C₅)alkylphenyl, H, O(C₁-C₅)alkyl, O(C₁-C₅)alkylphenyl and O-phenyl or alternatively wherein R₁ is methyl.

24. (canceled)

25. The compound of formula (V) as defined in claim 3, wherein R′″ is H; and R₁ is CH₃.
